(19) United States
(12) Patent Application Publication
(10) Pub. No.: US 2021/0302459 A1
(43) Pub. Date: Sep. 30, 2021

(54) Biosensor Storage Methods

(71) Applicant: Queen's University at Kingston, Kingston (CA)

(72) Inventors: Zhe She, Kingston (CA); Jennifer Ferrer McLeod, Kingston (CA)

(21) Appl. No.: 17/211,937

(22) Filed: Mar. 25, 2021

Related U.S. Application Data
(60) Provisional application No. 62/994,327, filed on Mar. 25, 2020.

(51) Int. Cl. G01N 37/00; G01N 27/327

(52) U.S. Cl. CPC G01N 37/00; G01N 27/3275

(57) ABSTRACT

A method for storing an electrochemical biosensor that includes disposing an electrochemical biosensor in buffer solution, and maintaining the electrochemical biosensor and buffer solution at a selected temperature, wherein the biosensor comprises at least two toll-like receptor proteins bound on a conductive surface, wherein the buffer comprises L-Arg, L-Glu, NaCl, imidazole, and Tris-HCl, the selected temperature is about 4° C., and pH of the buffer is about 8; or wherein the buffer comprises phosphate buffered saline, the selected temperature is about −80° C., and pH of the buffer is about 7.4.

### RELATED APPLICATION

This application claims the benefit of the filing date of U.S. Application No. 62/994,327, filed on Mar. 25, 2020, the contents of which are incorporated herein by reference in their entirety.

### FIELD

The invention relates to methods for storing biosensors.

### BACKGROUND

Bacteria detection and identification is important in environmental monitoring and clinical diagnostics. Detecting and reporting bacterial contaminants in water from public beaches is very important for public safety. Detection and identification currently are done using traditional methods, which include transportation of samples to a laboratory. The turn-around time for laboratory results is often from 2 to 5 days. Portable coliform detection systems for water analysis may provide total counts of bacterial cells within hours but they lack identification. Electrochemical transducers have been widely studied as portable methods for detecting bacterial cells and toxins. Electrochemical bacteria biosensors, which commonly are built with transducer and bio-recognition components, are capable of detection sensitivity of 1 cell/mL (see Neufeld, T. et al., Anal. Chem., 2003, 75, 580-585). However, storage conditions may degrade performance of such biosensors, reducing reliability and reproducibility of results.

### SUMMARY

In one embodiment, the invention provides a method for storing an electrochemical biosensor, including disposing an electrochemical biosensor in buffer solution, and maintaining the electrochemical biosensor and buffer solution at a selected temperature, wherein the biosensor comprises at least two toll-like receptor proteins bound on a conductive surface, wherein the buffer solution comprises L-Arg, L-Glu, NaCl, imidazole, and Tris-HCl, the selected temperature is about 4° C., and pH of the buffer is about 8, or wherein the buffer solution comprises phosphate buffered saline (PBS), the selected temperature is about −80° C., and pH of the buffer is about 7.4.

In one embodiment, the concentration of L-Arg is about 45 to about 55 mM. In one embodiment, the concentration of L-Arg is about 50 mM. In one embodiment, the concentration of L-Glu is about 45 to about 55 mM. In one embodiment, the concentration of L-Glu is about 50 mM. In one embodiment, the concentration of NaCl is about 0.9 to about 1.1 M. In one embodiment, the concentration of NaCl is about 1 M. In one embodiment, the concentration of imidazole is about 190 to about 210 mM. In one embodiment, the concentration of imidazole is about 200 mM. In one embodiment, the concentration of Tris-HCl is about 45 to about 55 mM. In one embodiment, the concentration of Tris-HCl is about 50 mM. In one embodiment, the biosensor detects gram-positive bacteria. In one embodiment, the electrochemical biosensor is for detecting gram-positive bacterial whole-cells. In one embodiment, the biosensor detects diacylated lipopeptide. In one embodiment, the electrochemical biosensor comprises an immune receptor. In one embodiment, the electrochemical biosensor is for detecting contaminants. In one embodiment, the electrochemical biosensor is for detecting contaminants in biological samples. In one embodiment, the electrochemical biosensor is for detecting gram-positive bacteria. In one embodiment, the electrochemical biosensor comprises toll-like receptor proteins 2 and 6. In one embodiment, the electrochemical biosensor is disposed on an electrode surface. In one embodiment, the conductive surface comprises gold, silver or glassy carbon.

In one embodiment, the invention provides a buffer for storing electrochemical biosensors, comprising L-Arg, L-Glu, NaCl, imidazole, and Tris-HCl, and pH of the buffer is about 8. In one embodiment, the invention provides a buffer for storing electrochemical biosensors, comprising phosphate buffered saline (PBS), and pH of the buffer is about 7.4.

In one embodiment, the invention provides a kit for storing electrochemical biosensors, comprising a buffer for storing electrochemical biosensors, that includes L-Arg, L-Glu, NaCl, imidazole, and Tris-HCl, and pH of the buffer is about 8; or phosphate buffered saline (PBS), and pH of the buffer is about 7.4.

### BRIEF DESCRIPTION OF THE DRAWINGS

For a better understanding of the invention and to show more clearly how it may be carried into effect, reference will now be made, by way of example, to the accompanying drawings, wherein:

FIG. 1A shows an illustration of TLR2/6 biosensor preparation and detection, including (a) Bare gold surface; (b) modification with the linker molecule, 1-lipoic acid n-hydroxysuccinimide ester; (c) immobilization of TLR2 and TLR6 proteins; (d) blocking unreacted linker molecules; (e) biosensor exposed to analytes.

FIG. 1B shows a schematic of a three step TLR2/6 biosensor preparation and detection, including a gold surface bearing representative linker molecules (e.g., 1-lipoic acid n-hydroxysuccinimide ester); immobilization of a mixture of TLR2 and TLR6 proteins; and blocking unreacted linker molecules with ethanolamine.

FIG. 2A shows a bar graph of detection responses obtained from TLR2/6 biosensors in the presence of 0.1 μM of Pam2CSK4 with no storage and with storage under different conditions for 2 weeks, the conditions were: A. fresh sensors; B. biosensors stored at 4° C. in PBS buffer; C. −33° C. in 50% v/v PBS/glycerol; D. −33° C. in 50% v/v Tris/glycerol; E. −80° C. in PBS; F. −80° C. without being in any solution; and G. 4° C. in amino acid solution.

FIG. 2B shows a bar graph of detection responses obtained from TLR2/6 biosensors in the presence of 1.0 μM of Pam2CSK4 with no storage and with storage under different conditions for 2 weeks, the conditions were: A. fresh biosensors; B. biosensors stored at 4° C. in PBS buffer; C. −33° C. in 50% v/v PBS/glycerol; D. −33° C. in 50% v/v Tris/glycerol; E. −80° C. in PBS; F. −80° C. without being in any solution; and G. 4° C. in amino acid solution.

FIG. 2C shows a bar graph of detection responses obtained from TLR2/6 biosensors in the presence of 50 μM of Pam2CSK4 with no storage and with storage under different conditions for 2 weeks, the conditions were: A. fresh biosensors; B. biosensors stored at 4° C. in PBS buffer; C. −33° C. in 50% v/v PBS/glycerol; D. −33° C. in 50% v/v Tris/glycerol; E. −80° C. in PBS; F. −80° C. without being in any solution; and G. 4° C. in amino acid solution.

FIG. 3A shows cyclic voltammetry measurements of bare gold surface (▪), surface after LPA modification (▴), surface after TLR modification (•), surface after blocking with ethanolamine (▾); the arrow shows a trend of the changes in voltammograms.

FIG. 3B shows square wave voltammograms measuring bare gold surface ( [symbol] ), surface after LPA modification (▴), surface after TLR modification (▪), surface after blocking with ethanolamine (•); the arrow shows a trend of the changes in voltammograms.

FIG. 3C shows a Nyquist plot having a y-axis of −Zⁿ and a x-axis of Z′, which are also known as imaginary resistance component (−Z_{Im}) and real resistance component (Z_{Re}), respectively, measuring bare gold surface (▾), surface after LPA modification (▴), surface after TLR modification (▪), surface after blocking with ethanolamine (•); the arrow shows a trend of the changes in the impedance plots.

FIG. 4 shows a bar graph comparison of TLR2/6 biosensor responses to different concentrations of Pam2CSK4 (left bars), and lipopolysaccharide (right bars).

FIG. 5A shows a plot of responses of TLR2/6 biosensors to different strains of bacterial whole-cell cultures (1) E. hirae; (2) B. licheniformis; (3) E. coli 25922 at a concentration of 100 CFU/mL.

FIG. 5B shows a plot of responses of TLR2/6 biosensors to different strains of bacterial whole-cell cultures (1) E. hirae; (2) B. licheniformis; (3) E. coli 25922 at a concentration of 10⁴ CFU/mL.

FIG. 5C shows a plot of responses of TLR2/6 biosensors to different strains of bacterial whole-cell cultures (1) E. hirae; (2) B. licheniformis; (3) E. coli 25922 at a concentration of 10⁶ CFU/mL.

FIG. 6A shows a calibration curve of different concentrations of Pam2CSK4 obtained from TLR2/6 biosensors stored for 2 weeks at 4° C. in PBS buffer, where ΔR_{CT}(%) is normalized detection signal.

FIG. 6B shows a calibration curve of different concentrations of Pam2CSK4 obtained from TLR2/6 biosensors stored for 2 weeks at −33° C. in 50% v/v PBS/glycerol buffer.

FIG. 6C shows a calibration curve of different concentrations of Pam2CSK4 obtained from TLR2/6 biosensors stored for 2 weeks at −80° C. in PBS buffer.

FIG. 6D shows an expanded view of a portion of FIG. 6C.

FIG. 6E shows an expanded view of a portion of FIG. 6A.

FIG. 6F shows an expanded view of a portion of FIG. 6B.

FIG. 7A shows electrochemical output from measuring different concentrations of Gram (+) bacteria, specifically a cyclic voltammogram.

FIG. 7B shows electrochemical output from measuring different concentrations of Gram (+) bacteria, specifically a square wave voltammogram.

FIG. 7C shows electrochemical output from measuring different concentrations of Gram (+) bacteria, specifically a Nyquist plot from electrochemical impedance measurements where curves plotted with (▪), (•), (▴), and (▾) are for increasing concentrations of B. licheniformis from 0, 10², 10⁴ to 10⁶ CFU/mL, wherein a continuous line represents a fitted curve of electrochemical impedance results that were calculated using the inset electronic circuit shown to extract R_{ct} detection signals and used for constructing a calibration curve in FIG. 7D.

FIG. 7D shows electrochemical output from measuring different concentrations of Gram (+) bacteria, specifically, a response to different concentrations of bacteria with an R² value of 0.95, wherein the average responses and error bars were obtained from 3 repeats using 3 individual prepared biosensors.

FIG. 7E shows electrochemical output from measuring different concentrations of Gram (+) bacteria, specifically, a cyclic voltammogram.

FIG. 7F shows electrochemical output from measuring different concentrations of Gram (+) bacteria, specifically, a square wave voltammogram.

FIG. 7G shows electrochemical output from measuring different concentrations of Gram (+) bacteria, specifically, a Nyquist plot from electrochemical impedance measurements where curves plotted with (▪), (•), (▴), and (▾) are for increasing concentrations of E. hirae from 0, 10², 10⁴ to 10⁶ CFU/mL, wherein the average responses and error bars were obtained from 3 repeats using 3 individual prepared biosensors, and a continuous line shows electrochemical impedance results that were analysed using an electronic circuit (inset) shown to extract R_{ct} detection signals, which were used for constructing a calibration curve in FIG. 7H.

FIG. 7H shows electrochemical output from measuring different concentrations of Gram (+) bacteria, specifically response to different concentrations of bacteria with an R² value of 0.96, wherein the average responses and error bars were obtained from 3 repeats using 3 individual prepared biosensors.

FIG. 8A shows a plot of electrochemical responses to different concentrations of Pam2CSK4 at 0.1, 0.5, 1.0, 5.0, 10.0, 50.0 μM. ΔR_{ct}(%)=[R_{ct} (concentration)−R_{ct} (control)]/R_{ct} (control), the error bars were calculated standard deviations based on 4 replicates, wherein each replicate represents one biosensor prepared and tested independently.

FIG. 8B shows an expanded view of a portion of FIG. 8A.

FIG. 9A shows a cyclic voltammogram of TLR2/6 sensors before and after exposure to different concentrations of Pam2CSK4, specifically 0.1, 0.5, 1.0, 5.0, 10.0, or 50.0 μM of Pam2CSK4 for 15 minutes each at a scan rate of 0.1 V·s⁻¹ and all measurements were obtained using a 10 mM HEPES aqueous buffer at pH 7.4, containing 5 mM/5 mM Fe(CN)₆^{3-/4-} as a redox couple and 1 M NaClO₄ as the supporting electrolyte.

FIG. 9B shows a square wave voltammogram of TLR2/6 sensors before and after exposure to different concentrations of Pam2CSK4, specifically 0.1, 0.5, 1.0, 5.0, 10.0, and 50.0 μM of Pam2CSK4 for 15 minutes each, wherein measurements were obtained using a 10 mM HEPES aqueous buffer at pH 7.4, containing 5 mM/5 mM Fe(CN)₆^{3-/4-} as a redox couple and 1 M NaClO₄ as the supporting electrolyte.

FIG. 9C shows Nyquist plots of TLR2/6 sensors before and after exposure to different concentrations of Pam2CSK4, specifically 0.1, 0.5, 1.0, 5.0, 10.0, and 50.0 μM of Pam2CSK4 for 15 minutes each, wherein measurements were obtained using a 10 mM HEPES aqueous buffer at pH 7.4, containing 5 mM/5 mM Fe(CN)₆^{3-/4-} as a redox couple and 1 M NaClO₄ as the supporting electrolyte; a continuous line represents a fitted curve of electrochemical impedance results that were calculated using the inset electronic circuit shown to extract R_{ct} detection signals, which were used for constructing a calibration curve in FIG. 8A and FIG. 8B.

### DETAILED DESCRIPTION OF EMBODIMENTS

One of the challenges in making and using biosensors is how to maintain the performance of the sensor after storage. Storage conditions are described herein that maintain the performance of biosensors that include at least two toll-like receptor proteins bound on a conductive (e.g., metal) surface. These storage conditions were shown to maintain the binding affinities of the biosensors' toll-like receptors to analytes. The storage conditions include temperature ranges and solution conditions for storage of protein biosensors that include receptors immobilized on a metal surface.

Determining storage conditions that provide stability of proteins immobilized on metal surfaces is important because maintaining activities of such immobilized molecules is different from maintaining activities of these molecules in solutions. Suitable storage conditions are essential for real-life applications of biosensors since these conditions maintain the ability of the biosensor detectors to be used after storage. Toll-like receptor (TLR)-based biosensors have attracted significant interest in providing broad-spectrum detection of bacterial contaminants. TLR biosensors are biosensors that include two types of TLR proteins that bind an analyte through formation of a dimer (e.g. TLR1/TLR1, TLR5/TLR5 for a homodimer, TLR1/TLR2, TLR2/TLR6 for heterodimers). Accordingly, the term “TLR2/6” used herein refers to a heterodimer of TLR2 and TLR6.

TLR biosensors provide a bio-recognition element that selectively recognizes pathogen-associated molecular patterns (PAMP), which are not associated with specific bacterial strains (Vasselon, T. et al., Infect. Immun., 2002, 70, 1033). For example, TLR4 detects lipopolysaccharides (LPS) in outer membranes of gram-negative bacteria cells regardless of the strains (Kawai, T. et al., Nat. Immunol., 2010, 11, 373-384). As described herein, a biosensor that recognizes gram-positive bacteria cells was prepared using at least two toll-like receptor proteins (e.g., TLR2 and TLR6) and hybridizing them onto sensor electrode surfaces (e.g., a metal surface). As described in Examples 3 and 4, detection of bacterial whole-cells by these biosensors was validated using Pam2CSK4, a gram-positive PAMP. The detection limits for two gram-positive cells, specifically B. licheniformis and E. hirae, were 10² CFU/ml and 10⁴ CFU/mL.

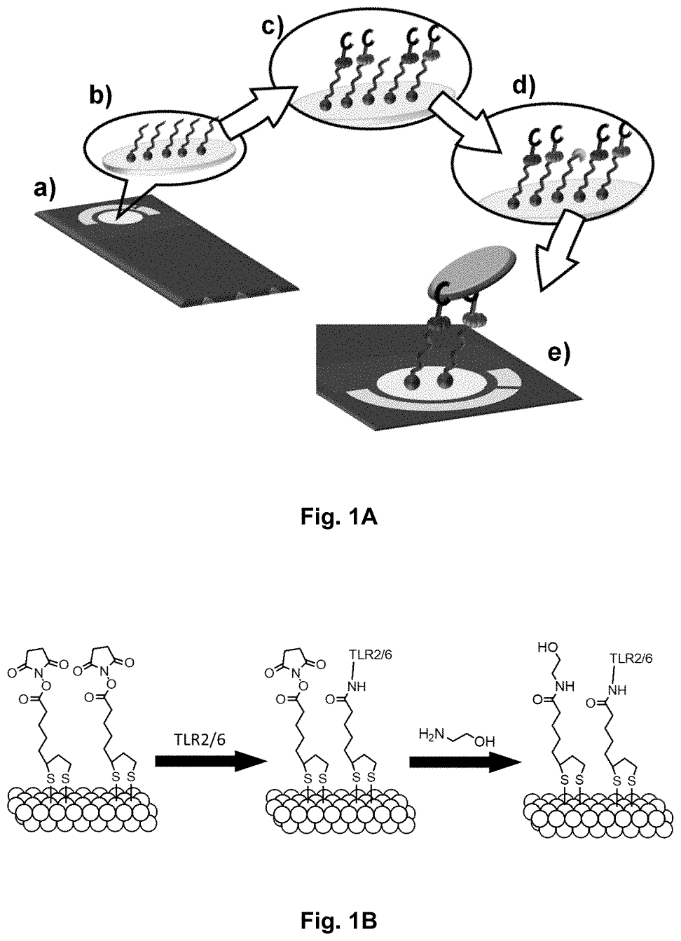

Referring to FIGS. 1A and 1B, an illustration and a schematic are shown for the steps of preparing a toll-like receptor biosensor for detecting PAMP from gram-positive bacteria. The proteins play essential roles in providing bio-recognition capability. The biosensor was constructed by immobilizing a mixture of TLR2 and TLR6 proteins on screen-printed electrode surfaces. The electrode surface is a conductive surface. Examples of conductive surfaces include glassy carbon and metal. Non-limiting examples of metal surfaces include gold and silver. The proteins are immobilized on the conductive surface through a linker molecule. A linker has a first end that attaches to the conductive surface, and a second end that attaches to the protein. A non-limiting example of a linker molecule is 1-lipoic acid n-hydroxysuccinimide ester (LPA). Isolated PAMPs and commercial bacterial cells were suspended as samples for testing. Cultured batches of bacterial whole-cells in broth solutions were used to validate the biosensors. Biosensor responses to freshly prepared bacterial samples were investigated.

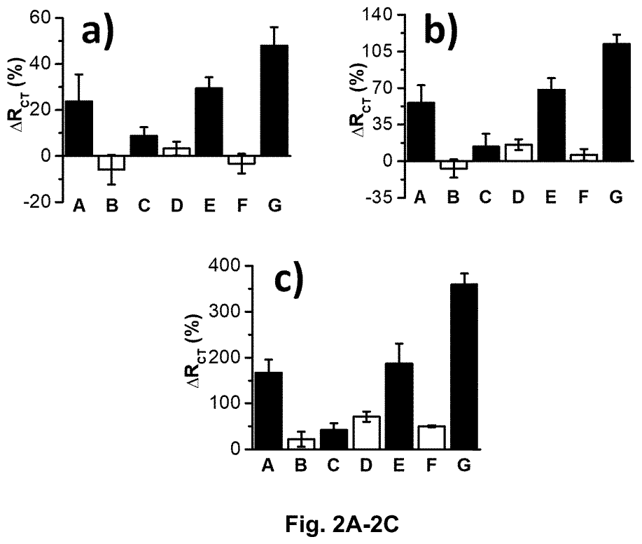

Referring to FIGS. 2A-C, plots are shown that provide detection responses obtained from TLR2/6 biosensors in the presence of Pam2CSK4 with no storage, and with storage under different conditions for 2 weeks (see Example 5 for details). Columns labelled ‘A’ in FIGS. 2A-C correspond to detection responses of fresh (i.e., not stored) TLR2/6 biosensors to different concentrations of Pam2CSK4. Responses of TLR2/TLR6 biosensors stored in PBS buffer at −80° C. (see columns labelled ‘E’ in FIGS. 2A-2C) for two weeks to Pam2CSK4 were equivalent to the responses from freshly prepared biosensors. That is, there was no significant loss of performance under storage condition E (see Table 1).

TABLE 1
Storage Conditions and Temperatures for Trials

| | | Storage |
|---|---|---|
| Batch | Storage Environment | Temperature |
| A | Fresh no storage (control) | |
| B | PBS buffer | 4° C. |
| C | 50% v/v PBS/glycerol | −33° C. |
| D | 50% v/v Tris/glycerol | −33° C. |
| E | PBS buffer | −80° C. |
| F | N₂ (g) container | −80° C. |
| G | Amino Acid Buffer | 4° C. |

In comparison to the fresh biosensors, TLR2/6 biosensors that were stored in an amino acid solution (see columns labelled ‘G’ in FIGS. 2A-2C) produced an enhanced detection signal. As shown in FIG. 2C, a response of 167±29% was observed for freshly prepared biosensors (column ‘A’), while the response from storage condition ‘G’ was 360±24% when exposed to the same concentration of Pam2CSK4. Although not wishing to be bound by theory, the inventors suggest that the reason for this enhanced detection signal is improvement of the performance of the biosensors due to better organized molecular layers and improved orientation for binding.

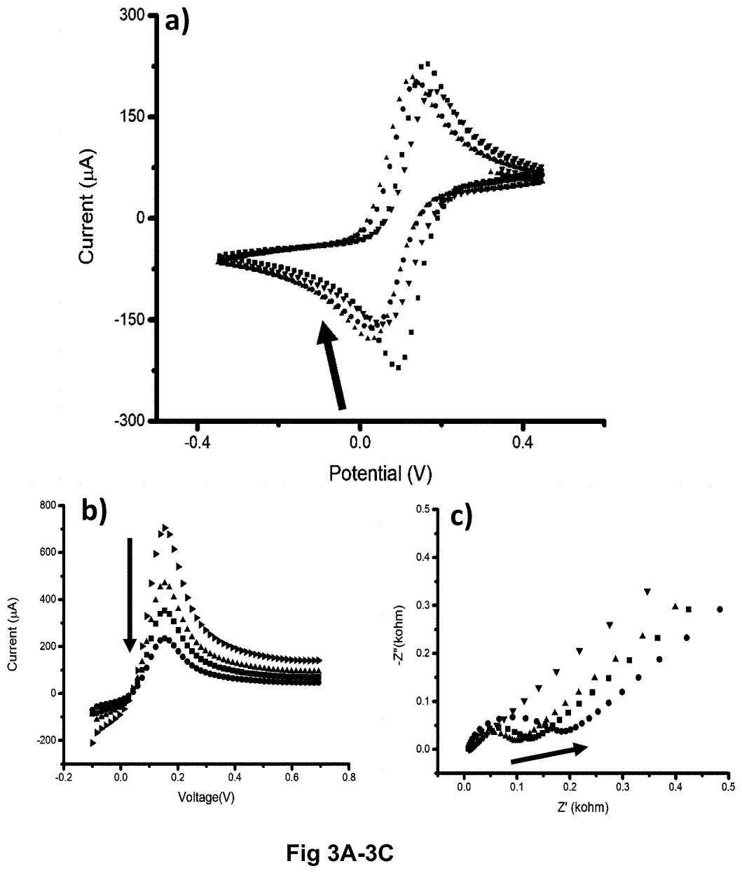

Referring to FIG. 3A-C, plots show characterization of biosensor surfaces during preparation steps. Arrows in FIGS. 3A and 3B demonstrate a decrease in measured current as the electrode surface is increasingly modified. That is, they show reduction of electrochemical currents after each step, which are due to reduced charge transfer by successful modifications. The arrow in FIG. 3C shows increasing surface resistance with increased modification of the surface, which consists of both imaginary (−Z″) and real (Z′) resistance components. Larger impedance and resistance leads to reduced charge transfer.

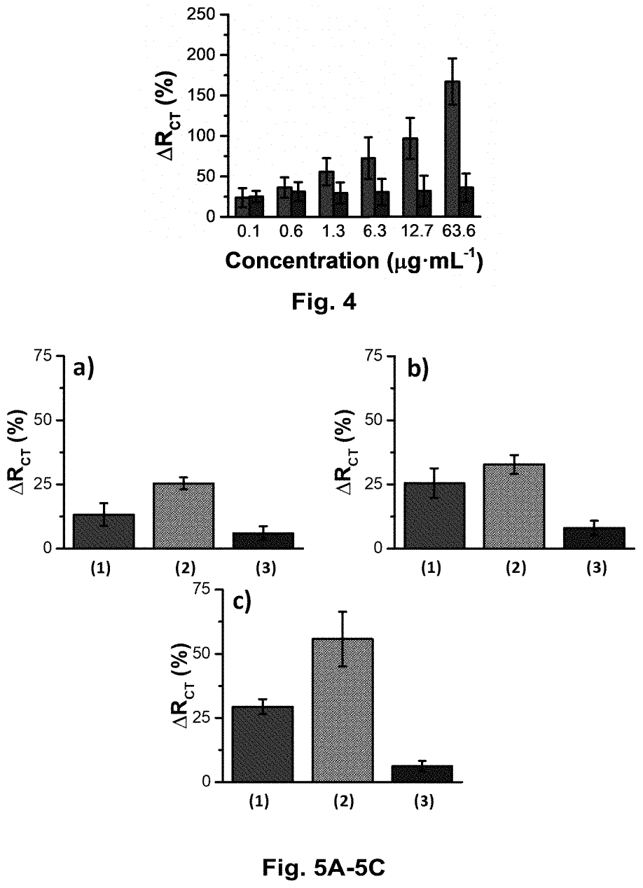

Referring to FIG. 4, bar graphs are shown that compare TLR2/6 biosensor responses to different concentrations of Pam2CSK4 (left bars), and lipopolysaccharide (right bars).

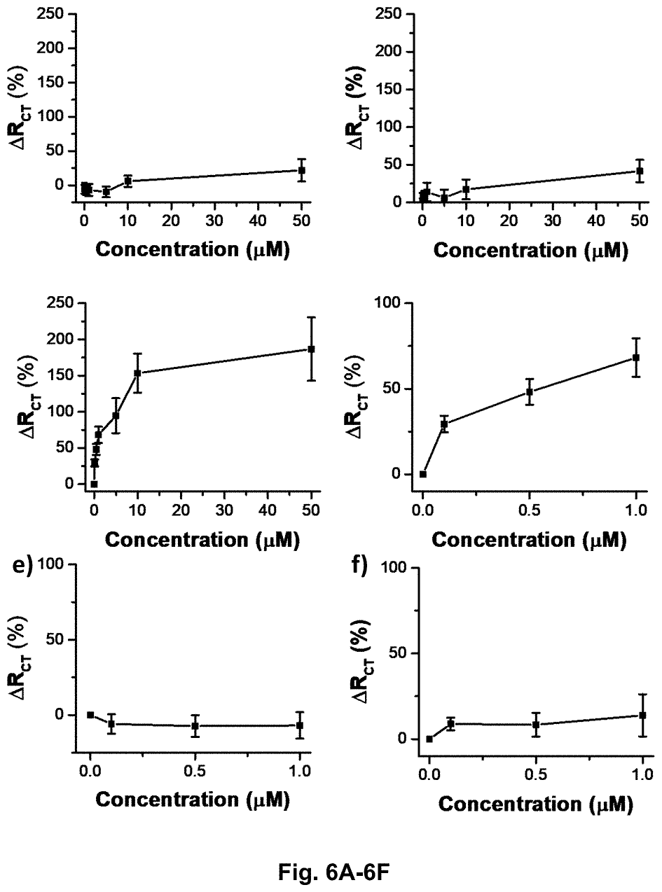

Referring to FIG. 5A-5C, a plot is shown of responses of TLR2/6 biosensors to different strains of bacterial whole-cell cultures (1. E. hirae; 2. B. licheniformis; 3. E. coli 25922) at varying concentration.

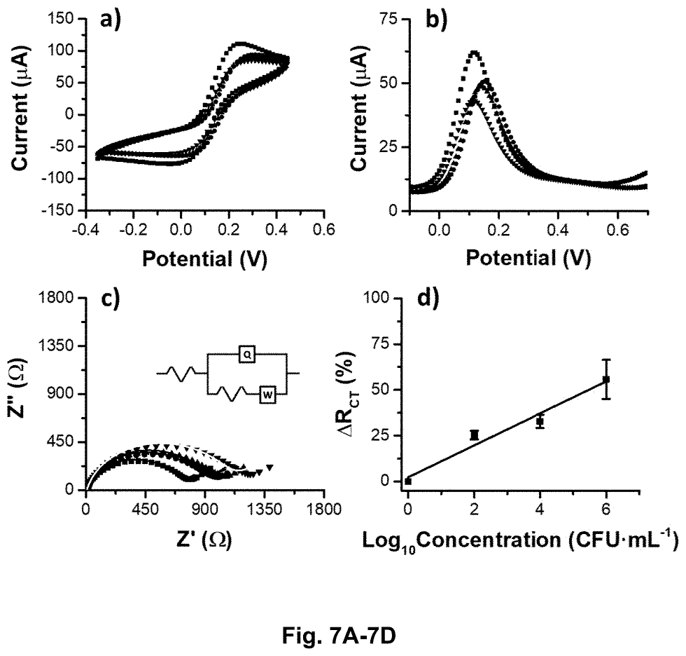

Referring to FIG. 6 A-D, calibration curves are shown of different concentrations of Pam2CSK4 obtained from TLR2/6 biosensors stored for 2 weeks in different conditions.

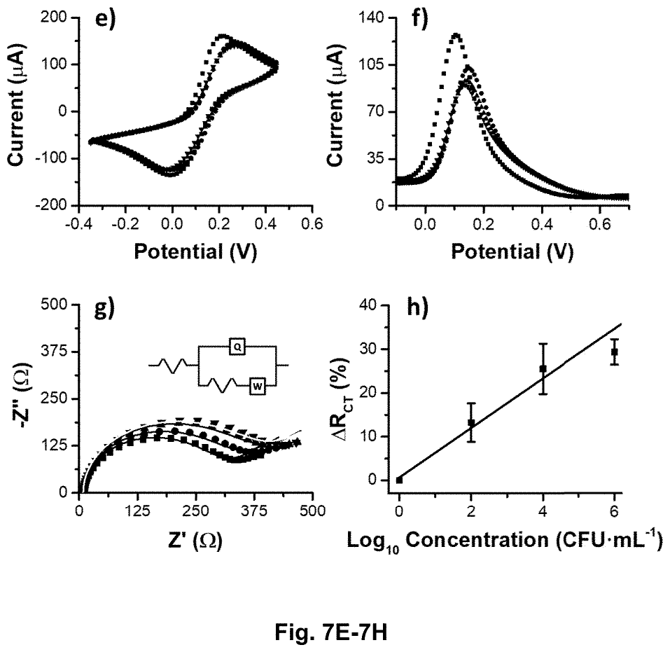

Referring to FIG. 7A-H, plots are shown of electrochemical output from measuring different concentrations of Gram (+) bacteria.

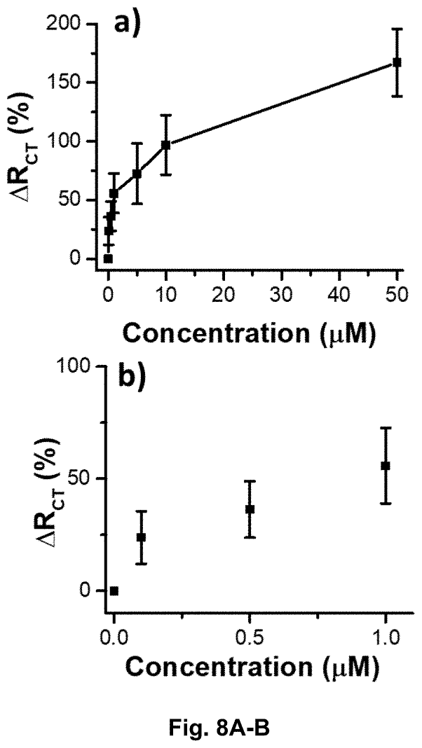

Referring to FIGS. 8A-B, plots are shown of electrochemical responses to different concentrations of Pam2CSK4.

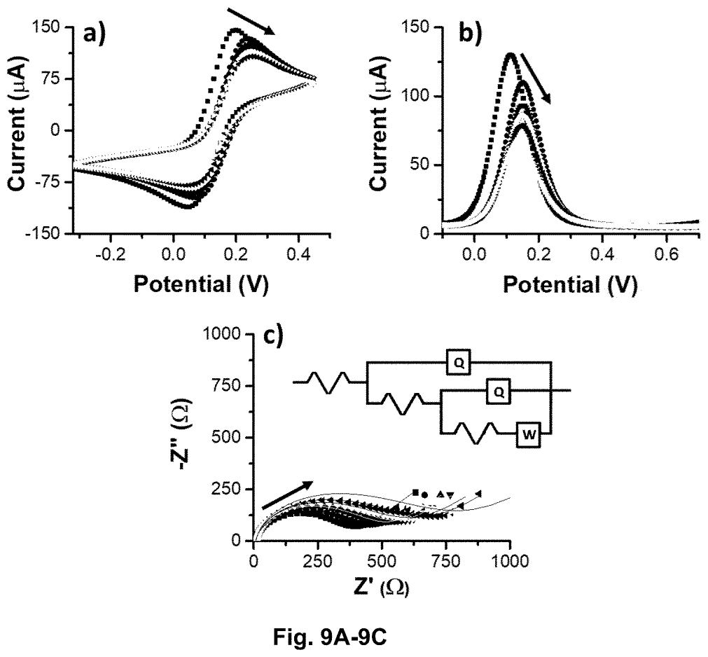

Referring to FIGS. 9A-9C, plots are shown of activity of TLR2/6 biosensors before and after exposure to different concentrations of Pam2CSK4.

In one embodiment, a kit for storage of an electrochemical biosensors is provided. The kit includes an amino acid buffer, or a PBS buffer, as described in Example 5. The kit may also include instructions, or information about a website with instructions for use of the kit.

The following working examples further illustrate the invention and are not intended to be limiting in any respect.

### WORKING EXAMPLES

### Materials and Methods

Anhydrous ethanol was purchased from Greenfield Global (Brampton, ON, Canada). (4-(2-hydroxyethyl)-1-piperazineethanesulfonic acid (HEPES) (99%), phosphate buffered saline (PBS) buffer (pH ˜7.4), potassium ferrocyanide trihydrate (98.5%), potassium ferricyanide (99%) sodium perchlorate monohydrate (laboratory grade) and hydrochloric acid (37%) were purchased from Fisher Scientific (Ottawa, ON, Canada). Tris base (99%) was purchased from TCI America (Portland, Oreg., USA). Ethanolamine (99%) was purchased from Alfa-Aesar (Ward Hill, Mass., USA). Sodium hydroxide (98%) was purchased from BioShop Canada Inc. (Burlington, ON, Canada). Toll-like receptor proteins, recombinant mouse TLR2 Fc chimera protein (1530-TR-050) and recombinant mouse TLR6 Fc Chimera protein (1533-TR-050) were purchased from R&D Systems Inc. (Minneapolis, Minn., USA). Pam2CSK4 was purchased from InvivoGen (San Diego, Calif., USA).

Milli-Q (MQ) water was obtained from a Millipore Synthesis A10 Milli-Q water system. pH measurements were carried out on a Fisherbrand pH probe. DRP-C220AT DropSens screen printed electrodes and DRP-CAC (model code) connectors were purchased from METROHM AG® (Herisau, CH). Electrochemical measurements were performed on a CHI6055E potentiostat purchased from CH Instruments (Austin, Tex., USA). All experiments were performed at room temperature under ambient conditions.

### Bacteria Preparation

Whole-cell bacteria was cultured in sterile 15 mL test tubes. 5 mL of Tryptone Soya Broth (TSB) for B. licheniformis and E. coli 25922 and Todd Hewitt Broth (THB) for E. hirae was added to each respective test tube. One bacterial culture from each respective strain was added to the appropriate test tube with a wooden inoculating stick, where the end containing the bacterial culture was broken off into the media. The test tubes were secured with a screw cap lid and placed in a shaking incubator at 37° C. for 18 hours. After 18 hours the cultures were placed in a refrigerator to inhibit further bacterial growth. A 10-fold serial dilution was carried out in sterile test tubes. The bacterial cultures were taken from the refrigerator and vortexed 3 times for three seconds each. 1 mL of the bacterial culture was then added to a test tube containing 9 mL of reverse osmosis (RO) water and vortexed 3 times for three seconds. 1 mL of the newly diluted solution was placed in another test tube containing 9 mL of RO water and vortexed, and this was repeated a predetermined number of times depending on the bacteria strain and growth. A predetermined volume (bacteria depending) of the lowest concentration was then dispensed onto a Tryptic Soy Agar (TSA) plate and spread over the surface with a sterile spreading instrument. The plate was then incubated overnight at 37° C. and the resulting colonies on the plate were counted allowing for the bacteria concentration to be calculated.

### Analyte Dilution

Pam2CSK4 solutions were prepared by serial dilution. 1.5 mL of MQ water was added to 1 mg of Pam2CSK4 and the resulting solution was mixed on a vortex machine for 2 minutes. A serial dilution was then carried out in HEPES buffer generating 0.1, 0.5, 1.0, 5.0, 10.0, and 50.0 μM solutions. Bacteria solutions were prepared by a serial dilution in HEPES buffer. The cultured whole-cell bacteria was diluted in HEPES buffer generating solutions with 1×10², 1×10⁴, and 1×10⁶ CFU/mL.

### Example 1. Preparation of TLR 2/6 Hybridized Biosensor

As shown in FIGS. 1A-B, a TLR2/6 biosensor was prepared following the steps as shown. Specifically, TLR biosensors were prepared using DropSens gold screen printed electrode (SPE). 1-lipoic acid n-hydroxysuccinimide ester (LPA, a linker molecule) was synthesized following a published protocol (see Howarth, M., et al., Nat. Methods, 2008, 5, 397-399). LPA solution at a concentration of 2 mM was prepared using ethanol/MQ water (volume ratio of 1:1). A clean plastic petri dish was lined with a filter paper which was dampened with a 1:1 solution of ethanol and MQ. The DropSens SPE was placed on the filter paper and 50 μL of the 2 mM LPA solution was dropped onto the working electrode. The petri dish was sealed with parafilm and placed in a fridge at 277 K for 24 hours. An additional 50 μL of the 2 mM LPA solution was cast onto a working electrode and returned to the fridge for another 24 hours. After a total of 48 hours the SPE was rinsed thoroughly with ethanol and MQ water before blown dry with nitrogen. TLR2 and TLR6 proteins were individually reconstituted into solutions at a concentration of 200 μg/mL using PBS buffer (pH ˜7.4). The TLR2/6 protein mixture was prepared by mixing 100 μL of TLR2 and 100 μL of TLR6 and mixing on a vortex machine for 30 seconds in a 1.5 mL microcentrifuge tube. The SPEs were placed in petri dishes with filter papers dampened with PBS buffer (pH ˜7.4). A small drop of TLR 2/6 mixture (5 μL) was dispensed onto each working electrode and then the petri dishes were sealed with PARAFILM™ and left in the fridge at 277 K for 72 hours. The electrodes were rinsed with MQ water after the immersion and blown dry with nitrogen. An ethanolamine-tris buffer solution was prepared by dissolving 1.2 g of ethanolamine, 0.121 g tris base in 20 mL of MQ water. The pH of the solution was adjusted to 8.4 using concentrated hydrochloric acid. After the step of immobilizing TLR2/6 proteins, electrode surfaces were immersed in ethanolamine solutions for 1 hour to deactivate unreacted ester groups on the LPA monolayer.

### Example 2. Electrochemical Measurements of Biosensors

Gold surfaces were monitored using cyclic voltammetry and electrochemical impedance spectroscopy. Oxidation and reduction peaks shown in FIG. 3A correspond to redox couple of Fe(CN)₆^{3-/4-}. Peak current was lowered as a gold surface of the electrode was modified with a linker and a protein molecule. Molecules on the gold surface reduced charge transfer between the gold electrode and the redox couple. Reduced charge transfer was also observed using impedance measurement. As shown in FIG. 3C, surface modification led to a larger arc in Nyquist plots. Electrodes with completed TLR2/TLR6 modifications were made into several batches for testing against different analytes and under different storage conditions.

All electrochemical measurements were preformed in faraday cages using DRP-CAC adapters for SPEs being measured. Cyclic voltammetry (CV), square wave voltammetry (SWV), and electrochemical impedance spectroscopy (EIS) measurements were completed using CHI6055E potentiostats (CH Instruments of Austin, Tex., USA). Measurements were carried out in 10 mM HEPES buffer solutions with 5 mM K₄Fe(CN)₆/5 mM K₃Fe(CN)₆ and 1 M NaClO₄ as the supporting electrolyte. CV measurements were carried out in a window between −350 and 450 mV using a scan rate of 0.1 V/s. SWV was performed by scanning from −100 mV to 700 mV with a frequency of 15 Hz. Open-circuit potentials were always used for EIS measurement, which were conducted in the frequency range of 100000 to 0.1 Hz with an amplitude of 5 mV. The experimental EIS curves were evaluated to determine the film resistance using ZSimpWin 2.0 software (available from AMETEK® Scientific Instruments of Berwyn, Pa., USA). The exposure times for Pam2CSK4 and bacterial whole-cell samples were 5 and 15 minutes respectively. CV, SWV and EIS were always collected before and after exposure of the biosensors to the analytes.

### Example 3. Biosensor Responses to Different Concentrations of a Gram-Positive PAMP

Sensor responses to different concentrations of Pam2CSK4, a gram-positive PAMP, were evaluated. Cyclic voltammograms were collected for the TLR2/6 biosensors before and after their exposures (see FIG. 9A). As the concentration of the Pam2CSK4 increased from 0.1 to 0.5, 1.0, 5.0, 10.0 and 50.0 μM, the peak current was reduced from 147 μA to 96 μA. A similar trend was observed using square wave voltammograms (see exemplary one in FIG. 9B). The current suppression from the voltammograms (FIGS. 9A and 9B) is indicative of surface coverage. Reduction in measured current correlated to the binding of Pam2CSK4 onto the TLR2/6 biosensor. Binding increased the amount of molecules on surfaces and blocked electron transfer between the electrode and Fe(CN)₆^{3-/4-}. Electrochemical impedance spectroscopy was also applied to measure the resistance of the molecules on surfaces. Nyquist plots (see FIG. 9C) showed an increase in the size of the arcs. A simple model (FIG. 9C inset) was used to produce simulated curves fitted in the experimental ones. The resistance for each concentration was evaluated.

A normalized calibration of electrochemical response (ΔRct %) versus concentration of Pam2CSK4 was plotted (see FIG. 8). The average for each value was obtained from 4 replicates. Each replication was carried out with one newly prepared TLR2/6 biosensor. The TLR2/6 biosensor has shown responses from 0.1 to 50.0 μM of Pam2CSK4. As shown in FIG. 8, responses to 0.1 μM and 1 μM of the PAMP were 24±12% and 58±13% respectively. The increase from 0.1 to 1 μM was quite linear (y=36x+20). The response trend to concentration was relatively less steep (y=5x+50) between 1 μM and 10 μM, suggesting the TLR2/6 binding sites on the biosensor surfaces were saturated at higher concentrations of Pam2CSK4 ligands. As TLR2/6 combination was known to recognize PAMPs in gram (+) bacteria rather than gram (−) bacteria, therefore a comparison experiment with lipopolysaccharide, a gram (−) PAMP was carried out. A batch of TLR2/6 biosensors was prepared by the same conditions. The biosensors were then exposed to different concentrations of LPS from 0.1 to 63.6 μg/ml. (Concentration by weight was used as the molecular weight of LPS varies.) Following the same experimental steps in electrochemical impedance spectroscopy and simulation by ZSimpWin, the responses were obtained (see FIG. 4). The comparison between Pam2CSK4 and the Gram (−) agonist (Salmonella LPS), demonstrated the specificity of this biosensor towards Gram (+) strains of bacteria. The response from the Salmonella LPS never exceeded ˜40% in comparison with Pam2CS4 with a response of ˜160% at the largest concentration. This experiment suggests that there is some non-specific binding between PAMPs and the biosensor surfaces. It was also observed that the responses triggered by non-specific binding flatten across different concentrations of LPS. In comparison, selective binding to Pam2CSK4 led to increased responses at higher concentrations.

After testing with isolated PAMPs as analytes, the response of TLR2/6 biosensors to two strains of whole-cell bacteria was studied (see FIGS. 7A-H). B. licheniformis and E. hirae are both gram-positive bacterial strains, while B. licheniformis has flagella on cell surfaces and E. hirae does not. Since flagella are in the scope of TLR5 but not TLR2/6, while diacylated lipopeptide (e.g. Pam2CSK4) is recognized by TLR2/6, it was expected that some responses from both strains would be observed.

One batch of TLR2/6 biosensors was exposed to different concentrations of B. licheniformis at 10², 10⁴, and 10⁶ CFU·mL⁻¹. CV, SWV and electrochemical impedance were applied to monitor the changes on biosensor surfaces as shown in FIG. 7A-C. After exposing the biosensor surface to B. licheniformis solution, the current was reduced from 62 μA (squares) gradually down to 43 μA (triangles). This current reduction indicated an increase in blockage of heterogeneous charge transfer between the gold electrode surface and Fe(CN)₆^{3-/4-} ions. A Nyquist plot (see FIG. 7C) shows that the size of the arcs increased as the concentration of B. licheniformis was increased. Replicated preparation and analysis were performed to construct a calibration curve (see FIG. 7D). The responses (ΔRct %) for concentrations of 10², 10⁴, and 10⁶ CFU·mL⁻¹ were 25±2%, 33±4% and 56±11% respectively.

Another batch of biosensors was used to detect different concentration of E. hirae whole-cells. A similar trend was observed as shown in FIGS. 7E-G. The resistance of biosensor surfaces was increased with increasing concentration. However, the amount of the responses was slightly different for E. hirae whole-cells. The responses (ΔRct %) for concentrations of 10², 10⁴, and 10⁶ CFU·mL⁻¹ were 13±4%, 26±6% and 29±3% respectively, which were consistently lower than the responses from B. licheniformis.

### Example 4. Testing of Biosensors Using a Gram-Negative Bacterial Strain

A comparison experiment was carried out exposing TLR2/6 biosensors to E. Coli 25922 whole-cell cultures, which is a gram-negative bacterial strain. FIGS. 5A-C shows a comparison between the two gram-positive bacterial strains and E. coli 25922 at consistent concentrations. As gram-negative bacteria (e.g. E. coli 25922) is not in the scope of TLR2/6 recognition, it should not trigger any responses to TLR2/6 biosensors in theory, but possibly due to unspecific binding 6±3% of changes observed for 10² CFU·mL⁻¹. The responses did not change when the concentrations of the E. coli 25922 were increased 100 and 10,000 folds, which were 8±3% and 6±2% for 10⁴ and 10⁶ CFU·mL⁻¹. For each concentration, student t-tests were performed on responses from gram-negative bacterial samples and one of the gram-positive bacterial samples. The responses from E. hirae and E. coli 25922 at 10² CFU·mL⁻¹ were not quite significantly different (p-value was 0.07). For all the other concentrations, the responses from E. coli 25922 were significantly different from the gram-positive bacteria strains at the same concentration (p-value ranged from 0.0004 to 0.01). This result showed that detection by TLR2/6 biosensors was highly selective towards gram-positive bacterial strains. It was thus possible to differentiate responses at 10² CFU·mL⁻¹ for B. licheniformis and at 10⁴ CFU·mL⁻¹ for E. hirae.

### Example 5. Storage of Biosensors

Biosensors as described herein (e.g., TLR2/6 proteins immobilized onto conductive surfaces) were stored under varying conditions (see Table 1) for two weeks and then challenged by the PAMP (Pam2CSK4). As shown in FIGS. 2A-2C, a comparison was done of the responses from freshly prepared and stored biosensors were compared. Calibration curves including more concentration points are shown in FIGS. 6A-6D, where ΔR_{CT}(%) is a normalized detection signal. ΔR_{CT}(%)=[R_{CT} (after exp.)−R_{CT} (before exp.)]/R_{CT} (before exp.), where “before exp.” refers to before exposure to analyte, and “after exp.” refers to after exposure to the analyte.

Three storage temperatures were selected, 4° C., −33° C., and −80° C. Ionic salt solutions are commonly used to maintain protein folding and stability, thus, to preserve the biosensor during storage, varying buffer solutions were used.

Batch B biosensors were stored in PBS buffer of pH˜7.4 at 4° C. As shown in FIGS. 2A-2C, there were no significant responses from the TLR2/6 biosensors after 2 weeks storage for Batch B.

The storage conditions for Batch C was −33° C. in 50% v/v PBS buffer at pH ˜7.4 and glycerol. The storage conditions for Batch D was −33° C. in 50% v/v Tris buffer pH ˜7.7 and glycerol. Initially Batches C and D had a very small response to Pam2CSK4 with no change in signal between the two smaller concentrations, 0.1 and 1.0 μM but at the highest concentration (50 μM) there was a response. However, the activity was reduced down to 30% in comparison to control.

The storage conditions for Batch E was −80° C. in PBS buffer at pH ˜7.4. The response for all concentrations measured were within error of the control biosensor. Batch E storage conditions thus demonstrated no loss in biosensor activity.

Batch F biosensors were stored at −80° C. in the absence of any buffer solution. As shown in FIGS. 2A-C, Batch F biosensors lost most of their response.

Batch G biosensors were disposed in a buffer solution that included amino acids. Specifically, Batch G buffer solution included 50 mM L-Arg, 50 mM L-Glu, 1 M NaCl and 200 mM imidazole in 50 mM Tris-HCl (pH˜8.0). The solution surrounded each biosensor in Batch G and they were kept at 4° C.

Different concentrations of Pam2CSK4 were used to trigger the responses. These concentrations were 0.1 μM for FIG. 2A; 1.0 μM for FIG. 2B; and 50 μM for FIG. 2C.

### EQUIVALENTS

It will be understood by those skilled in the art that this description is made with reference to certain embodiments and that it is possible to make other embodiments employing the principles of the invention which fall within its spirit and scope.

We claim:

1. A method for storing an electrochemical biosensor, comprising:
disposing an electrochemical biosensor in buffer solution; and
maintaining the electrochemical biosensor and buffer solution at a selected temperature;
wherein the biosensor comprises at least two toll-like receptor proteins bound on a conductive surface,
wherein the buffer solution comprises L-Arg, L-Glu, NaCl, imidazole, and Tris-HCl, the selected temperature is about 4° C., and pH of the buffer is about 8; or
wherein the buffer solution comprises phosphate buffered saline (PBS), the selected temperature is about −80° C., and pH of the buffer is about 7.4.

2. The method of claim 1, wherein the concentration of L-Arg is about 45 to about 55 mM.

3. The method of claim 2, wherein the concentration of L-Arg is about 50 mM.

4. The method of claim 1, wherein the concentration of L-Glu is about 45 to about 55 mM.

5. The method of claim 4, wherein the concentration of L-Glu is about 50 mM.

6. The method of claim 1, wherein the concentration of NaCl is about 0.9 to about 1.1 M.

7. The method of claim 1, wherein the concentration of imidazole is about 190 to about 210 mM.

8. The method of claim 8, wherein the concentration of imidazole is about 200 mM.

9. The method of claim 1, wherein the concentration of Tris-HCl is about 45 to about 55 mM.

10. The method of claim 10, wherein the concentration of Tris-HCl is about 50 mM.

11. The method of claim 1, wherein the biosensor detects gram-positive bacteria.

12. The method of claim 1, wherein the biosensor detects diacylated lipopeptide.

13. The method of claim 1, wherein the electrochemical biosensor comprises an immune receptor.

14. The method of claim 1, wherein the electrochemical biosensor is for detecting contaminants.

15. The method of claim 1, wherein the electrochemical biosensor is for detecting gram-positive bacteria.

16. The method of claim 1, wherein the electrochemical biosensor comprises toll-like receptor proteins 2 and 6.

17. The method of claim 1, wherein the electrochemical biosensor is disposed on an electrode surface.

18. The method of claim 1, wherein the conductive surface comprises gold, silver or glassy carbon.

19. A buffer for storing electrochemical biosensors, comprising: L-Arg, L-Glu, NaCl, imidazole, and Tris-HCl and pH of about 8; or phosphate buffered saline (PBS) and pH of about 7.4.

20. A kit for storing electrochemical biosensors, comprising a buffer for storing electrochemical biosensors that comprises L-Arg, L-Glu, NaCl, imidazole, and Tris-HCl, and has a pH of about 8; or a buffer for storing electrochemical biosensors that comprises phosphate buffered saline (PBS), and has a pH of about 7.4.
